(19) United States
(12) Patent Application Publication
(10) Pub. No.: US 2005/0101882 A1
(43) Pub. Date: May 12, 2005

(54) Safety pressure device for body fluid extraction

(76) Inventors: Enrique Carlos Leira, St. Louis, MO (US); Sridhar Condoor, St. Louis, MO (US); Michael Germani, Inver Grove Heights, MN (US); David Peso, Madrid (ES)

Correspondence Address: SAINT LOUIS UNIVERSITY;OFFICE OF RESEARCH ADMINISTRATION, 3556 CAROLINE STREET, ST. LOUIS, MO, 63104, US

(21) Appl. No.: 10/706,322

(22) Filed: Nov. 12, 2003

(51) Int. Cl. A61B 5/00

(52) U.S. Cl. 600/579

(57) ABSTRACT

Lumbar punctures (LP) are potentially hazardous when the cerebrospinal fluid (CSF) pressure is elevated. CT is not always helpful to anticipate elevated intracranial pressure and may not be available each time a LP must be preformed. Disclosed is a simple pressure-locking valve which is attachable to an LP needle. The pressure-locking valve closes off the flow of fluid when the pressure of the fluid exceeds a value which can be preset to the pressure-locking valve.

### BACKGROUND OF THE INVENTION

(1) Field of the Invention

The field of invention relates to devices and methods for safely sampling body fluids by preventing the removal of body fluids that are under excessive levels of pressure

(2) Description of the Related Art

Medical practitioners often need to invasively extract fluid from within a body. Commonly, blood, muscle compartment, and spinal column fluid are taken to assist in the diagnosis or treatment of patients. Practioners often need to access the venous system for placing catheters. An inadvertent puncture to the arterial system, which runs at a higher pressure than the veins, could result in blood loss unless the inadvertent puncture is rapidly detected. For those with neurological problems, a medical practitioner often performs a procedure, called the Lumbar Puncture (“LP”) (a.k.a. a spinal tap) to invasively enter the spinal or cerebrospinal fluid compartment, withdraw some spinal fluid for analysis, and measure the pressure within the spinal column. This procedure removes cerebrospinal fluid from within the spinal column for evaluation and measures the cerebrospinal fluid (CSF) pressure to assist in the diagnosis of various nervous system and medical conditions.

The current invasive implement used in LP is a small needle (a.k.a., cannula or needle cannula). The needle is inserted between the lumbar vertebrae just below the termination of the spinal cord, piercing the dura and reaching the fluid filled sub-dural space in the spinal column. Once located in this space, CSF flows into the distal tip of the needle and out the proximal end of the needle. The proper location of the needle is ascertained by this outflow of CSF.

A stopcock and open tube manometer may be connected to the proximal end of the needle. With the stopcock in the open position, the manometer fills with CSF until its elevation (Pressure Head) equals the pressure inside the spinal column. Equalizing the pressure can take between 5 and 15 minutes. This elevation is expressed in pressure units (e.g. mmHg or cmH₂O) and is known as the patient's opening pressure. After this pressure measurement, some of the fluid contents of the manometer can then be removed through a release valve into a collection vial for analysis, such as in the diagnosis for spinal meningitis or hemorrhage.

Once a sufficient quantity of CSF has been removed for analysis (typically 9 to 15 ml) the manometer can be re-filled as above (an additional 5 to 15 minutes). Once stabilized, this new pressure measurement on the manometer is said to be the patient's closing pressure. If the difference between the opening and closing pressures is large, this may indicate a spinal blockage that prevents the normal auto-regulation function that should compensate for the loss of fluid and maintain a constant CSF pressure. Many physicians skip this step of measuring closing pressure to save time even though this step is important in assessing the patient's condition. If a blockage is diagnosed by a large drop in CSF pressure, then the removal of an excessive amount of CSF may cause harm to the patient, i.e., if too much fluid is removed the pressure gradient between the brain and spinal column may cause herniation of the brain structures and damage to the brain stem and death. Lumbar punctures (LP) are potentially hazardous when the cerebrospinal fluid (CSF) pressure is elevated. Although controversial, computerized tomography (CT) scans are often used prior to the procedure to help identify patients at risk for herniation. Unfortunately, CT is not universally available and a normal study does not always exclude elevated intracranial pressure (ICP), such as in children with meningitis. In such situations, the elevated pressure will only be detected after a certain amount of CSF is lost within the manometer. Since such CSF loss can potentially increase the risk of herniation, stopping further pressure measurement after the column of fluid in the manometer reaches 200 mm of H₂O has been suggested. In addition, there are concerns about the level of awareness of some medical trainees to the risks of CSF removal in case of elevated pressure. All these scenarios stand to benefit from a safety valve at the needle opening that would automatically prevent fluid removal at a pre-specified CSF pressure value.

There have been attempts to use alternative detection systems rather than manometer systems in the measuring of body fluid pressures, such as Tempkin et al., U.S. Pat. No. 3,610,228, Moriuchi et al., U.S. Pat. No. 4,790,193, Davis et al., U.S. Pat. No. 4,817,629 and Williams, U.S. Pat. No. 5,935,083. Each of those referenced devices requires either a complex valve switching system, a chamber that must be filled with a fluid, an external device for priming, or an electronic measuring device with corresponding limitations on usefulness due to complexity and potential cost restrictions. Consequently, these alternative devices which measure CSF pressure at the needle port never gained clinical acceptance.

The simple innovative device described herein employs a pressure sensitive valve that shuts off the flow of body fluid in the event the pressure exceeds a preset value during the above exemplified invasive procedures. This simple and inexpensive invention may be used with the already existing stopcock and manometer arrangement or other downstream ancillary device, as part of a body fluid extraction procedure. The pressure measurement of body fluids during the execution of the LP and other body fluid extraction procedures will continue to be possible with the existing manometers. The simple device described in this application instead provides a safe and effective method to shut off the flow of body fluids when the pressure of such fluids are in excess of a preset range, above which pressure the removal of fluids may be detrimental to the patient.

### SUMMARY OF THE INVENTION

The Device

The invention provides a simple device, which can be placed at the proximal end (base) of a needle used for the extraction of a fluid from an area. The instant device can impede the flow of fluid when the pressure in the area sampled exceeds a predetermined pressure range. Once this predetermined pressure range is exceeded the instant device will block the flow of the fluid through the instant device. A preferred fluid is a body fluid, such as, for example, blood or cerebral spinal fluid (“CSF”).

In a first aspect depicted in FIGS. 1, 1A, 2, and 2A, the device comprises a housing, a fluid channel which communicates between an inlet opening and an outlet opening, and a movable rod in a rod sleeve which can block the communication between the inlet and the outlet depending on the pressure of the fluid at the inlet. The inlet allows the body fluid delivered from a needle to communicate with the outlet opening via the fluid channel situated between the inlet and the outlet. The rod in this first aspect has a generally cylindrical shape. The rod either allows the fluid to flow through the fluid channel or blocks flow through the fluid channel at a preset pressure. The rod in this particular aspect is sized such that the body fluid cannot flow past the rod in the rod sleeve, but can flow through the fluid channel. When incoming body fluid enters the fluid channel it contacts the rod in the rod sleeve such that when pressures at or exceeding a preset value the rod is displaced in the rod sleeve, occludes the fluid channel, and blocks the flow of the fluid out of the housing through the outlet opening. Preferably, the device has a housing and an inlet to which a permanent or removable needle can be attached.

In a second aspect depicted in FIGS. 3, 4 and 5, the device comprises a housing, a fluid channel which communicates between an inlet opening and an outlet opening, and a rod which can block the communication between the inlet and the outlet. The inlet allows the fluid delivered from a needle to communicate with the outlet opening via the fluid channel situated between the inlet and the outlet. The rod in this aspect is in a conical shape and sized such that the body fluid is not allowed to flow past the rod in the straight portion of the flow channel but must flow through the rod via channels in the rod. The rod is attached to the outlet of the housing via a spring that retards the motion of the rod in the direction of flow while the pressure is below a preset value (preferably 200 mm of H₂O) and allows the body fluid to flow unimpeded through the rod via the rod channels in the rod and to the outlet of the housing. When the body fluid pressure is at or above a preset value the rod is displaced toward the outlet of the housing and closes off the outlet, thereby blocking the flow of fluid out of the housing. In a preferred embodiment, the device has a housing and an inlet to which a permanent or removable needle can be attached.

In a third aspect depicted in FIGS. 6, 7 and 8, a device similar to the device of the second aspect is contemplated wherein the fluid channel extends straight through the housing with the rod in line with the channel (“inline”). The rod in this particular aspect is generally in a cylindrical or ingot shape sized such that the body fluid can not flow past the rod in the straight portion of the flow channel but must flow through the rod via at least one rod channel in the rod. The rod may be attached to the outlet of the housing via a spring that retards the motion of the rod in the direction of flow while the pressure is below a preset value and allows the fluid to flow unimpeded through the rod via the rod channel and to the outlet of the housing. When the pressure of the fluid pressure is at or above a preset value the rod is displaced toward the outlet of the housing and the rod channel is closed off, thereby blocking the fluid flow through the rod and to the outlet of the housing.

The instant device, as described in any one of its several preferred aspects and other aspects contemplated in the practice of this invention, may be operated at various differential pressures ranging from less than 10 mm of H₂O to greater than 200 mm of H₂O. Any needle, which may be used in conjunction with this invention, may be removable or permanently attached to the inlet of the instant device. The outlet of the instant device can be attached to an ancillary device, such as, for example, a tubing, a stopcock and manometer assembly, a three-way valve or other collection device. The tubing, stopcock and manometer assembly, three-way valve or other collection device attached to the outlet of the instant device can be either removable or permanently affixed. The fluid collected through the instant device may be a body fluid, such as, for example, a cerebral spinal fluid (“CSF”) or blood.

The Method

In another embodiment, the invention provides a method of extracting body fluid from a body area via a needle connected to a pressure sensitive valve permitting extraction of the body fluid when the body fluid pressure is below a preset value. When body fluid pressures in the body area are at or in excess of the preset value of the pressure sensitive valve, extraction of the body fluid is not allowed. This shutting off of body fluid flow at a preset value ensures greater patient safety while still providing for the extraction of the body fluid.

Although the instant device (supra) may be used to extract numerous types of body fluids, such as central venous lines (to prevent loss in case of an unintentional arterial puncture) muscle compartment edema, and CSF, the preferred instant method is described in the context of extracting CSF in a traditional LP procedure. In terms of an LP procedure the initial methodology comprises attaching a needle to the inlet of the instant device. Obviously, if the device employed has a needle permanently affixed to the inlet, the step of attaching a needle to the inlet is redundant and therefore unnecessary. Optionally, a tubing, a stopcock and manometer assembly, a three-way valve, a collection device or any combination of these items or other ancillary devices may be attached to the outlet of the instant device. Preferably, a three-way valve is attached to the outlet of the instant device further to which a stopcock manometer assemble is attached. According to the preferred LP method, the distal end (tip or point) of the needle is inserted between the lumbar vertebrae just below the termination of the spinal cord, piercing the dura and reaching the fluid filled sub-dural space in the spinal column. Once located in this space, CSF flows from the needle tip and out the proximal end (base) of the needle and to the inlet of the instant device.

When the pressure at the tip of the needle is preferably below 200 mm of H₂O, the flow of CSF from the inlet, through the fluid channel and to the outlet of the instant device is unrestricted. In a preferred embodiment, the three-way valve may be aligned to either: i) block the flow of CSF out of the instant device, ii) allow flow of the CSF to the stopcock manometer assembly whereby a measurement of the actual pressure present can be ascertained; or iii) allow flow out of the three-way valve to another ancillary device for collection or sampling. When the pressure is preferably at or above 200 mm of H₂O, the flow of body fluid from the inlet to the outlet through the fluid channel is cutoff, due to the displacement of the rod in, into or across the fluid channel of the instant valve such that the CSF cannot flow through the outlet.

The Kit

In yet another embodiment, the invention is drawn to a diagnostic kit, such as a LP kit, which comprises a needle and a pressure sensitive locking device of the instant invention (such as the device described supra). A preferred kit comprises a pressure sensitive locking device of the instant invention, a needle with stylus (1 inch or 1.5 inch 22 gauge or one 1 inch 25 gauge), a collection container or vial, antiseptic swab, gauze pad, towel, fenestrated drape and spot bandage.

Diagnostic kits, based on the described invention, will provide for the safe extraction of body fluid from a body area via a needle connected to a pressure sensitive valve which allows extraction of body fluid only under conditions in which the body fluid pressure is below a preset value. Although the preferred embodiment of the kit may be used to extract numerous types of body fluids, such as blood, muscle compartment edema, and CSF, the preferred instant kit will be described in the context of extracting CSF in a traditional LP procedure. Thus, the preferred embodiment of the kit comprises a needle to be attached to the inlet of the instant device, the instant device, a three-way valve, and a stopcock manometer assembly which may be attached to the outlet of the instant device. The three-way valve may be aligned to either: i) block the flow of CSF out of the instant device, ii) allow flow of the CSF to the stopcock manometer assembly whereby a measurement of the actual pressure present can be ascertained; or iii) allow flow out of the three-way valve to another ancillary device for collection or sampling.

### BRIEF DESCRIPTIONS OF THE DRAWINGS

FIG. 1 shows a top view of device comprising a rod sleeve, showing the rod and fluid channel in the open position.

FIG. 1A shows a top view of device comprising a rod sleeve, showing the rod and fluid channel in the closed position.

FIG. 2 is a side view of the housing in FIG. 1.

FIG. 2A is a side view of the housing in FIG. 1A.

FIG. 3 is top view of device with a conical inline rod.

FIG. 4 is a side view of the housing in FIG. 3.

FIG. 5 is a cross section of the rod from FIGS. 3 & 4.

FIG. 6 is top view of a device with a generally cylindrical with conical proximal (outlet) end.

FIG. 7 is a side view of the housing in FIG. 6.

FIG. 8 is a cross section of the rod from FIGS. 6 & 7.

FIG. 9 is a photograph of the prototype device.

FIG. 10 depicts the pressure values and response times required to activate the safety valve prototype for different inclinations

### DESCRIPTION OF THE PREFERRED EMBODIMENT

It is understood that this invention is not limited to the particular embodiments and methodologies, as these may vary. It is also to be understood that the terminology used herein is for the purpose of describing particular embodiments only, and is not intended to limit the scope of the present invention, which will be limited only by the appended claims.

Unless defined otherwise, all technical and scientific terms used herein have the same meanings as commonly understood by one of ordinary skill in the art to which this invention belongs. The preferred methods, devices, and materials are now described, although any methods and materials similar or equivalent to those described herein can be used in the practice or testing of the present invention. Nothing herein is to be construed as an admission that the invention is not entitled to antedate such disclosure by virtue of prior invention. Each reference cited herein is incorporated by reference herein in its entirety.

Definitions

The term “housing” means the external portion of the instant device in which the fluid channel is contained. Additionally the housing is a solid material in which the fluid channel is surrounded.

The term “needle” means a slender hollow instrument for introducing material into or removing material from the body. The term “needle” is equivalent to the term “cannula”.

The term “inlet” means an opening in the housing for fluids to enter. Preferably, it is the portion of the housing to which a needle may be attached to allow fluid to flow from a body area through the needle and into the housing.

The term “outlet” means an opening for fluid to exit. Preferably it is the portion of the housing to which an ancillary device may be attached to allow fluid to flow from a body area through the needle attached to the inlet and out of the housing.

The term “fluid channel” means a hollow, generally cylindrical section through the housing and through which fluid can flow.

The term “rod” herein refers to an, embolus, cone or cylinder shaped piece whereby when displaced is capable of blocking the flow of fluid through a fluid channel.

The term “obstructs the communication” means to block the flow of fluid between the inlet and the outlet.

The term “pressure differential” means the difference in pressure between two points in a system.

The term “preset value” means the numerical value at which a prescribed action will or will not take place. Preferably “preset value” means the pressure differential between inlet and outlet wherein the rod is displaced to block the flow of fluid in the fluid channel.

The term “displaced” means to actuate the rod in a direction that will either allow or curtail flow of fluid to the outlet.

The term “embolus” means a machined device that is capable of either allowing fluid flow or curtailing fluid flow based on its position in a fluid channel. In some aspect the “embolus” is equivalent to the “rod”.

The term “spring” means a device of a set tension that maintains the pressure on a rod such that the rod is not displaced when the fluid pressure is below a preset value and allows displacement when the fluid pressure is at or above a preset value.

The term “retaining pin” means a device to secure a second device to a fixed point in order to maintain the second device in a fixed position. As used herein the “retaining pin” is used to attach the spring to a fixed point on the housing.

The term “rod channel” means an opening through a rod that allows fluid to pass through the distal end of the rod and out through the proximal end of the rod or to pass through the proximal end of the rod and out through the distal end of the rod.

The term “rod sleeve” means a tubular section designed to fit the rod through which the rod can articulate.

The term “constriction” means any narrowing of a fluid channel restricting the flow of fluids. Preferably, “constriction” means any reduced cross sectional area along a portion of the length of the rod such that when the reduced cross sectional area of the rod is in fluid channel fluid flows to the outlet and when the reduced cross sectional area of the rod is not in the fluid channel fluid does not flow to the outlet.

The term “body fluid” means blood, lymph, CSF or other fluid contained within a bodily structure.

The term “contiguous with the fluid channel” means directly adjacent to, or continuous, with the fluid channel. Specifically the hollow, generally cylindrical section designed to allow fluid to flow.

The term “stopcock” means a valve assembly that can block the flow of fluid from one point to another point., usually a quarter turn valve.

The term “manometer assembly” means a device for measuring differential pressure.

The term “body area” means any potion of the body that is a likely candidate for the extraction of fluids.

The term “tubing” means a flexible device which can hold or convey fluid from one point to another point.

The term “pressure sensitive valve” means a device that is set to open or close based on a preset pressure value.

Embodiments

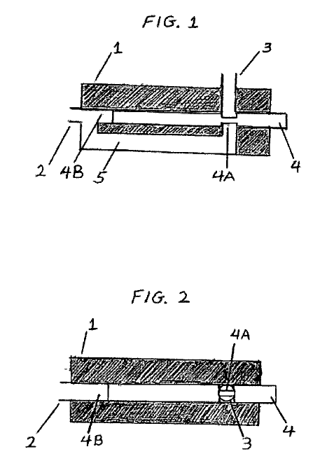

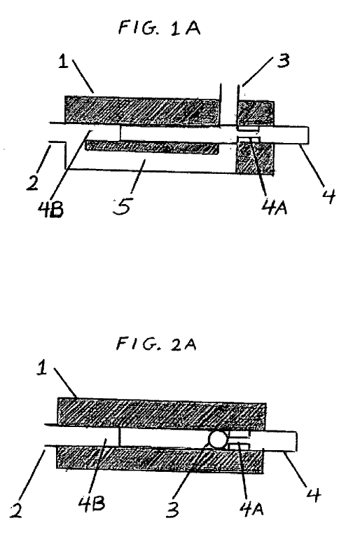

Disclosed is a device that acts as a pressure sensitive valve, which shuts off the flow of fluid whenever the fluid pressure is at or greater than a preset value. A preferred embodiment of the present invention, which shows the pressure sensitive valve in the open position is shown in FIGS. 1 and 2, with the pressure sensitive valve in the closed position shown in FIGS. 1A and 2A. This device has a housing 1 and an inlet 2 to which a permanent or removable needle can be attached. The needle's fluid channel extends from the tip end of the needle through to the housing 1, and connects with the inlet 2 communicating with the fluid channel 5. The fluid channel 5 extends through the housing 1 and allows communication between the inlet 2 and outlet 3. The housing also contains a rod 4 in a rod sleeve 4B that is sensitive to the incoming pressure of the fluid. When the pressure is below a preset value the flow of fluid from the inlet 2 to the outlet 3 through the fluid channel 5 is unrestricted. The fluid can flow from the inlet 2 through the fluid channel 5 past the reduced diameter portion 4A (“constriction”) of the rod 4, which is not displaced, and out through the outlet 3. According to the same preferred embodiment when the pressure is at or above a preset value the flow of fluid from the inlet 2 to the outlet 3 through the fluid channel 5 is restricted and the fluid cannot flow from the inlet 2 through the fluid channel 5 and out through the outlet 3. The restriction in flow at pressure at or above a preset value is caused by the rod 4, being displaced in the rod sleeve 4B such that the full size diameter portion (i.e. not the constriction) of the rod 4 is placed into the fluid channel 5 blocking the flow of fluid from the inlet 2 through the fluid channel 5 to the outlet 3 for the extraction of CSF is preferably about 200±20 mm of H₂O.

Although the above described embodiment may be used to extract numerous types of body fluids, such as blood, muscle compartment fluid, and CSF the use of this embodiment is described below for extracting CSF by way of example. However the skilled artisan in the practice of this invention will reasonably expect the instant device to be used in multiple other applications. A removable needle from a LP kit is attached to the inlet 2 of the housing 1, and the stopcock manometer assembly from the LP kit is attached to the outlet 3 of the housing. At this point in the LP procedure the practitioner inserts the needle into the lower spinal area of the patient. The lower lumbar spine (usually between the vertebrae known as L4-5) is preferable because the spinal cord stops near L2, and a needle introduced below this level will miss the spinal cord and encounter only nerve roots, which are easily pushed aside. The practioner inserts a needle in the space between two vertebrae of the lower back and slowly advances it toward the spine. A steady flow of clear CSF, normally the color of water, will begin to fill the needle as soon as it enters the spinal canal. The spinal fluid flows from the tip of the needle to the inlet 2 of the housing 1 entering the fluid channel 5 of the housing 1. When the pressure is below about 200±20 mm of H₂O (the preset value for the LP procedure) the flow of body fluid from the inlet 2 to the outlet 3 through the fluid channel 5 is unrestricted and CSF flows to the stopcock manometer assembly provided with the LP kit and a measurement of the actual pressure present can be ascertained. When the pressure is at or above about 200±20 mm of H₂O the flow of body fluid from the inlet 2 to the outlet 3 through the fluid channel 5 is restricted, due to the displacement of the full size diameter portion of the rod 4 into the fluid channel 5 such that the spinal fluid cannot flow through the outlet 3 into the stopcock manometer assembly provided with the LP kit.

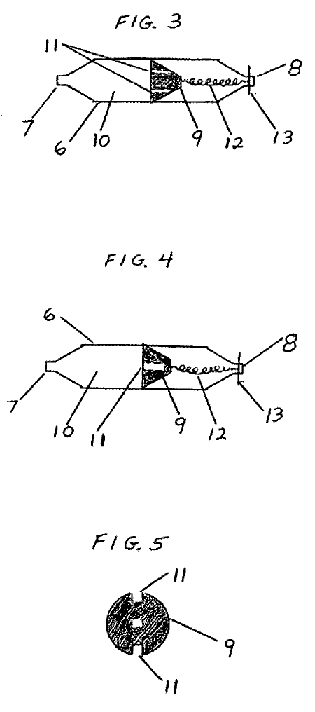

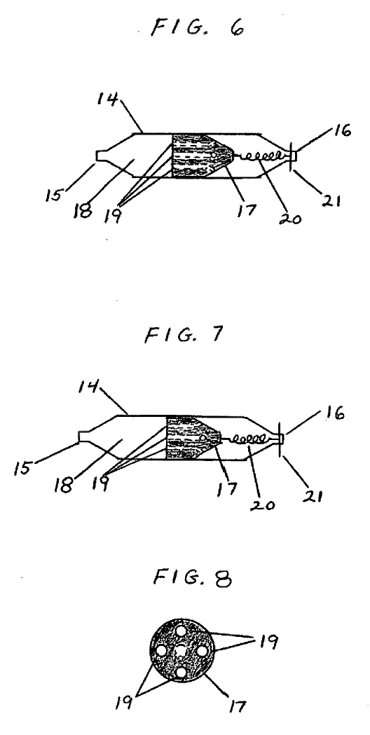

FIGS. 3, 4, and 5 depict an alternate embodiment of the invention. This device has a housing 6 and an inlet 7 to which a permanent or removable needle can be attached. The needle's fluid channel extends from the tip end of the needle through to the housing 6, and connects with the inlet 7 communicating with the fluid channel 10. In this embodiment the fluid channel 10 extends straight through the housing 6 with the rod 9 in line with the fluid channel 10. The rod 9 in this embodiment has a conical shape and sized such that the fluid is not allowed to flow past the rod 9 in the straight portion of the flow channel 10 but must flow through the rod 9 via a rod channel 11 integral to the rod 9. The rod 9 is attached to the outlet 8 of the housing 6 via a spring 12 and retaining pin 13 that retards the motion of the rod 9 in the direction of fluid flow while the pressure is below a preset value and allows the fluid to flow through the rod 9, via the rod channel 11 and out through the outlet 8 of the housing 6 unimpeded. When the fluid pressure is at or above a preset value the rod 9 is displaced toward the outlet 8 of the housing 6 and closes off the rod channel 11 integral to the rod 9 curtailing fluid flow out of the housing 6 through the outlet 8. A preferred preset value is between 10 and 390 mm of H₂O inclusively, with a more preferable preset value between 50 and 350 mm of H₂O inclusively, with an even more preferable preset value between 100 and 300 mm of H₂O inclusively, and with a most preferable preset value of about 200±20 mm of H₂O.

FIGS. 6, 7, and 8 depict yet another alternate preferred embodiment. This device has a housing 14 and an inlet 15 to which a permanent or removable needle can be attached. The needle's fluid channel extends from the tip end of the needle through to the housing 14, and connects with the inlet 15 communicating with the fluid channel 18. The rod 17 in this aspect is in a cylindrical conical shape sized such that the fluid is not allowed to flow past the rod 17 in the straight portion of the flow channel 18 but must flow through rod 17 via the rod channel 19 integral to the rod 17. The rod 17 is attached to the outlet 16 of the housing 14 via a spring 20 and retaining pin 21 that retards the motion of the rod 17 in the direction of flow while the pressure is below the preset value and allows the fluid to flow through the rod 17, via a rod channel 19, and out through the outlet 16 of the housing 14 unimpeded. When the fluid pressure is at or above the preset value the rod 17 is displaced toward the outlet 16 of the housing 14 and closes off the rod channels 19 integral to the rod 17 curtailing fluid flow out of the housing 14 through the outlet 16. A preferred preset value is between 10 and 390 mm of H₂O inclusively, with a more preferable preset value between 50 and 350 mm of H₂O inclusively, with an even more preferable preset value between 100 and 300 mm of H₂O inclusively, and with a most preferable preset value of about 200±20 mm of H₂O.

Materials of construction for the individual components of the instant device (i.e. the housing, rod, spring and retaining pin) of any embodiment preferably include those materials that can provide the structural integrity, allows for sterilization conditions (e.g. heat, steam, autoclave, chemical treatment, and irradiation) frictional and mass requirements. Preferably, the material of construction for the individual components is medical grade polyethylene. However, the skilled artisan in the manufacture of this invention will reasonably expect the instant device to be constructed of any material which will meet the structural, frictional and sterilization criteria discussed while allowing the instant device to work in the manner described in any embodiment of the instant device.

Although the preferred embodiments discussed above relate to sampling and measuring spinal fluid pressure, the invention is not so limited. While various embodiments and applications of this invention have been shown and described, it is apparent to those skilled in the art modifications are possible without departing from the inventive concepts herein. The invention, therefore, is not to be restricted except in the spirit of the appended claims.

### EXAMPLE

### Prototype Device

Summary

Lumbar punctures (LP) are potentially hazardous when the cerebrospinal fluid (CSF) pressure is elevated. Unfortunately, CT does not always allow one to anticipate elevated intracranial pressure or may not be available. Therefore, a simple pressure-locking valve attached to the LP needle could be useful to prevent CSF loss and prevent herniation. We built a prototype of a safety valve using plexiglass. The prototype housing had an interior flow line. A rod was designed to translate within the housing. Whenever the pressure at the needle opening exceeds the pre-designed value of about 200±20 mm of H₂O, the rod translated thereby locking the exit port and preventing further fluid flow through the valve. Laboratory performance tests were conducted as well as a feasibility experiment with a live animal. The prototype consistently responded to hydrostatic pressures higher than about 200±20 mm of H₂O by blocking the outflow of fluid within 0.5-1 seconds. In addition, the prototype worked in a realistic animal scenario. This prototype device may help minimize an uncommon but potentially lethal problem by preventing external fluid removal in high-pressure situations. It is safe, simple and inexpensive, so it could become an optional addition to the LP technique in situations associated with increased risk of herniation such as bacterial meningitis or unavailability of neuroimaging studies.

Medical Background

Lumbar punctures (LP) are potentially hazardous when the cerebrospinal fluid (CSF) pressure is elevated(1). Although controversial(2), computerized tomography (CT) scans are often used prior to the procedure to help identify patients at risk for herniation(3). Unfortunately, a normal CT does not always exclude elevated intracranial pressure (ICP), such as in children with meningitis(4), and is not universally available. In such situations, the elevated pressure will only be detected after a certain amount of CSF is lost filling the manometer tubing. Since such CSF loss can potentially increase the risk of herniation, it has been suggested to stop further pressure measurement after the column of fluid in the manometer reaches 200 mm(1). In addition, there are concerns about the level of awareness of some medical trainees to the risks of CSF removal in case of elevated pressure(1). All these scenarios stand to benefit from a safety valve at the needle opening that would automatically prevent fluid removal at a pre-specified CSF pressure value. While several alternative devices to measure CSF pressure have been proposed(5-8), they were too complex and therefore never gained clinical acceptance. Therefore, there is a need for a simple and feasible automatic safety pressure valve that could be attached to the LP needle opening.

Methods

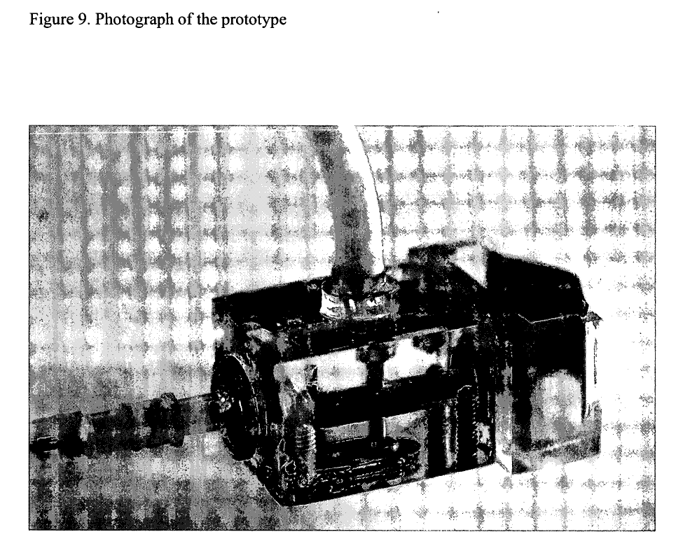

The prototype device selected for initial development, which is disclosed in this example uses differential pressure between the cerebrospinal fluid in the flow line and the atmosphere outside to allow/disallow flow. The schematic of the valve and the rod are shown in the FIGS. 1, 1A, 2, and 2A. In addition, FIG. 9 shows a photograph of the prototype attached to the LP needle.

During the normal operating conditions, the differential pressure on the rod (a.k.a. the rod) results in a small actuation force. The resisting frictional force balances this force. Thus, the hole or constriction in the rod is aligned with the flow line. When the differential pressure exceeds the pre-designed (preset) value of 200 mm of H₂O, the actuating force overcomes the resisting force, causing the rod to move to a new position. The hole in the rod becomes misaligned with the flow line. As a result, it shuts-off the fluid flow. Because this particular prototype valve is transparent, the operator could readily visualize the “locked” status. The operator can force open the valve by depressing the rod back into the open position.

The prototype was first tested in the laboratory by attaching it to the exit port of a horizontal 20-Gauge spinal needle. The needle-prototype device complex was kept in the horizontal plane while puncturing plastic tubing with a known and modifiable degree of hydrostatic pressure. Since the amount of pressure required to displace the rod depends in this case on the angle of inclination, performance was also tested for different needle inclinations to determine maximum allowable angle variations from the horizontal plane (FIG. 9).

In order to test the feasibility of the prototype on a live model, tests were conducted on a pig after obtaining the approval of the Saint Louis University Animal Care Committee. Anesthesia was induced with an intramuscular injection of TKX, and a mixture of tiletamine, ketamine, zolazepam and xylazine. Following induction, the trachea was intubated using a 6.5 mm cuffed endotracheal tube and the anesthesia was deepened and continued using isoflurane in oxygen. Dorsal-ventral radiographs of the lumbar spine and pelvis were obtained with markers to facilitate the LP. Surgical isolation of the left and right external and internal jugular veins using corresponding left and right paramedian neck incisions were performed to expose the vessels in case a ligation was needed to artificially increase the intracranial pressure (“ICP”). A central venous catheter was surgically implanted into the right external jugular vein for fluid and drug administration. The pig was then rotated to sternal recumbancy to permit lumbar puncture using guide marks established by radiography. Anesthesia was monitored using capnography, agent analysis and peripheral blood pressure as well as manual assessment of muscle tone and response to noxious stimulus. In addition to low levels of isoflurane in oxygen, supplemental anesthesia was provided by intermittent intravenous administration of TKX and ketamine.

Results

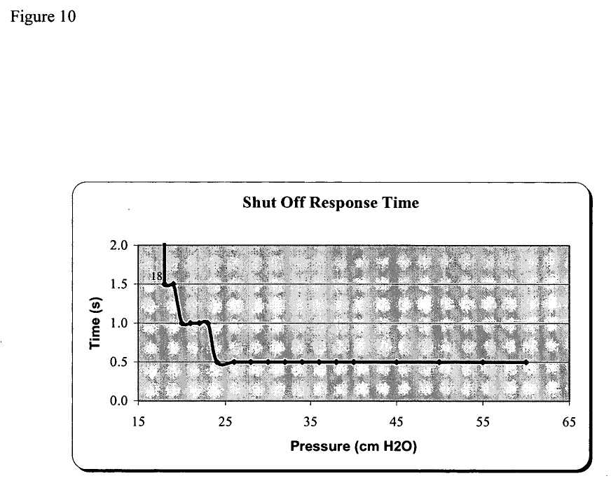

As shown in FIG. 10, in the laboratory testing the device prototype promptly and consistently responded to hydrostatic pressures higher than about 200±20 mm of H₂O by blocking the outflow of fluid. The prototype also performed within ±5 degrees of variation from the horizontal plain for this type of material. The condition for this that the valve must be kept at the same level as the insertion point of the needle. This is very hard to do because when the valve is angled it will naturally be higher/lower then when it is flat. When you move the valve up/down you are changing two factors, pressure and the resisting force. Gravity fights the rod when angled up, and the pressure is lower because the valve is above the insertion point. When angled down the opposite occurs. Preferably, the stainless steel will be replaced with a much lighter medical-grade plastic (i.e. medical grade polyethylene). While not intending to disclaim the use of stainless steel in the manufacture of this device, and wherein stainless steel may be used in the manufacture of this device, it is not considered to be a material of first choice. When this device is professionally manufactured stainless steel will not be the material of first choice due to weight and expense considerations. This will also serve to decrease the effects of gravity on the angled situations described and increase the angular range of the device.

In addition, the pig experiment proved that the prototype worked in a live scenario. After attaching the prototype device to the needle, the flowing CSF pressure immediately locked the valve suggesting that the opening pressure in that animal was at least about 200±20 mm of H₂O, and therefore preventing external CSF loss.

Discussion

This safety pressure valve prototype device was successful in blocking the outflow of cerebral spinal fluid whenever the pressures exceed the preset value, therefore preventing external fluid loss. The prototype device in this example requires only approximately 0.5 cc to fill up the valve instead of approximately 1.5 cc to fill up the manometer thereby increasing the safety of LP in high ICP settings when using this device. The concern of CSF extraction in cases of elevated ICP exists in medical practice(1;3). We offer a simple solution to address this already established concern.

The prototype device presented in this example operates as an on-off safety valve that does not actually measure pressure. But this should not interfere with the usual pressure measurement techniques of an LP. For example, in a low-pressure (open status) situation, pressures may be measured with the column manometer attached to the valve exit port. Conversely, in a high-pressure situation (blocked status) it would be at the discretion of the operator whether to continue further pressure measurement based on the clinical context. If further measurement is deemed necessary, the operator could remove the valve from the needle port and connect the tube manometer instead. A limitation of the prototype described in this orientation is that this valve only works within small variations of the horizontal plane, but this should not be a problem because the needle is kept horizontal during the LP procedure. However, other embodiments of the valve described above may work at any angle (e.g. those embodiments comprising a spring). Thos embodiments comprising a spring are particularly useful for LP work performed under fluoroscopy, wherein the LP needle is used in the vertical position.

The invention described herein is expected to reduce a potentially lethal problem. Because it is simple and inexpensive, the device could be easily manufactured as a disposable addition to lumbar puncture kits. Also, because the device is external to the body and uses sterilizable materials, no risks to the subjects are anticipated. This safety valve device could therefore become an important addition to the LP technique in situations at increased risk for herniation due to increased ICP, which includes cases of suspected meningitis, or whenever neuroimaging capabilities are not available. It could also be used as a warning device to increase the awareness of increased ICP among trainees.

The following references are cited throughout this disclosure using the associated numerical identifiers. Applicant makes no statement, inferred or direct, regarding the status of these references as prior art and reserves the right to challenge the accuracy of any statement made in these references. These references are incorporated herein by reference.

### REFERENCES CITED IN EXAMPLE

(1) Sharp C G, Steinhart C M. Lumbar puncture in the presence of increased intracranial pressure: the real danger. Pediatric Emergency Care 1987; 3(1):39-43.
(2) Archer B D. Computed tomography before lumbar puncture in acute meningitis: a review of the risks and benefits[comment]. [Review] [20 refs]. CMAJ Canadian Medical Association Journal 1993; 148(6):961-965.
(3) van Crevel H, Hijdra A, de Gans J. Lumbar puncture and the risk of herniation: when should we first perform CT?. [Review] [67 refs]. Journal of Neurology 2002; 249(2):129-137.
(4) Rennick G, Shann F, de Campo J. Cerebral herniation during bacterial meningitis in children. BMJ 1993; 306(6883):953-955.
(5) Williams P A. Device for Body Fluid Pressure Measurement. U.S. Pat. No. 5,935,083. 1999.
(6) Tempkin et al. Apparatus for Measuring Body Fluid Pressure. U.S. Pat. No. 3,610,228. 1971.
(7) Moriuchi et al. Pressure Transducer Apparatus. U.S. Pat. No. 4,790,193. 1988.
(8) Davis et al. Process and Apparatus for Measurement and Monitoring of Muscle Compartment Pressure. U.S. Pat. No. 4,817,629. 1989.

1. A device for collecting a fluid comprising a pressure sensitive valve, wherein said valve comprises:
(a) a housing;
(b) an inlet to the housing;
(c) an outlet from the housing;
(d) a fluid channel which can allow a fluid to flow from the inlet to the outlet; and
(e) a rod which can obstruct the communication between the inlet and the outlet, such that (i) when a pressure differential between the inlet and the outlet is at or greater than a preset value, the rod is displaced to obstruct the fluid from flowing from the inlet to the outlet and (ii) when the pressure differential between the inlet and the outlet is less than the preset value, the rod is not displaced to allow the fluid to flow from the inlet to the outlet.

2. The device of claim 1 wherein the rod is an embolus, cone, or cylinder.

3. The device of claim 1 wherein the rod is connected to the outlet by a spring and retaining pin.

4. The device of claim 1 wherein the rod is perpendicular to the fluid channel and in a rod sleeve.

5. The device of claim 1 wherein the rod is inline with the fluid channel.

6. The device of claim 1 wherein the rod comprises a constriction.

7. The device of claim 5 wherein the rod comprises a rod channel which is contiguous with the fluid channel.

8. The device of claim 1 wherein the preset value is at or greater than 10 mm of H₂O.

9. The device of claim 1 wherein the preset value is at or greater than 50 mm of H₂O.

10. The device of claim 1 wherein the preset value is at or greater than 100 mm of H₂O.

11. The device of claim 1 wherein when preset value is between 179 mm H₂O and 221 mm H₂O.

12. The device of claim 1 wherein the fluid comprises a body fluid.

13. The device of claim 1 wherein a needle is affixed to the inlet to the housing.

14. The device of claim 1 wherein a stopcock and manometer assembly is affixed to the outlet from the housing.

15. The device of claim 1 wherein a three-way valve is affixed to the outlet from the housing.

16. The device of claim 1 wherein a tubing is affixed to the outlet from the housing.

17. A method of extracting a body fluid from a body area, the method comprising the steps of:
(a) connecting a needle to an inlet of a housing of a valve, wherein the valve comprises the housing, the inlet of the housing, an outlet of the housing, a fluid channel which can allow a body fluid to flow from the inlet to the outlet, and a rod which can obstruct the body fluid from flowing from the inlet to the outlet, such that (i) when a pressure differential between the inlet and the outlet is at or greater than a preset value, the rod is displaced to obstruct the body fluid from flowing from the inlet to the outlet, and (ii) when the pressure differential between the inlet and the outlet is less than the preset value, the rod is not displaced to allow the body fluid to flow from the inlet to the outlet;
(b) inserting the needle into a body area, wherein the body fluid flows through the needle and into the inlet to the housing such that (i) when the body fluid is at a pressure that is below the preset value, the body fluid flows to the outlet of the housing to be collected, and (ii) when the body fluid is at a pressure that is at or in excess of the preset value, the rod is displaced and body fluid is not allowed to flow out of the outlet of the housing.

18. The method of claim 17 wherein the rod is an embolus, cone, or cylinder.

19. The method of claim 17 wherein the rod is connected to the outlet of the housing by a spring and retaining pin.

20. The method of claim 17 wherein the rod is perpendicular to the fluid channel and in a rod sleeve.

21. The method of claim 17 wherein the rod is inline with the fluid channel.

22. The method of claim 20 wherein the rod comprises a rod channel which is contiguous with the fluid channel.

23. The method of claim 17 wherein the body fluid comprises CSF or blood.

24. The method of claim 17 wherein a needle is affixed to the inlet of the housing.

25. The method of claim 17 wherein a stopcock is affixed to the outlet of the housing and a manometer is affixed to the stopcock.

26. The method of claim 17 wherein a three-way valve is affixed to the outlet of the housing.

27. The method of claim 17 wherein a tubing is affixed to the outlet of the housing.

28. A kit for extracting a body fluid from a body, the kit comprising a needle, a valve, and an ancillary device, and wherein,
(a) the valve comprises a housing, an inlet to the housing, an outlet from the housing, a fluid channel which can allow a body fluid to flow from the inlet to the outlet, and a rod which can obstruct the body fluid from flowing from the inlet to the outlet, such that (i) when a pressure differential between the inlet and the outlet is at or greater than a preset value, the rod is displaced to obstruct flow of the body fluid from the inlet to the outlet; and (ii) when the pressure differential between the inlet and the outlet is less than the preset value the rod is not displaced, which thereby allows flow of the body fluid from the inlet to the outlet;
(b) and the ancillary device is selected from the group consisting of a tubing, a three-way valve, a stopcock and manometer assembly and a collection device.

29. The kit of claim 28 wherein the rod is an embolus, cone, or cylinder.

30. The kit of claim 28 wherein the rod is connected to the outlet by a spring and retaining pin.

31. The kit of claim 28 wherein the rod is perpendicular to a portion of the fluid channel and in a rod sleeve.

32. The kit of claim 28 wherein the rod is inline with the fluid channel.

33. The kit of claim 31 wherein the rod comprises a rod channel which is contiguous with the fluid channel.

34. The kit of claim 28 wherein the body fluid comprises CSF or blood.

35. The kit of claim 28 wherein the needle is affixed to the inlet of the housing.

36. The kit of claim 28 wherein a stopcock and manometer assembly is affixed to the outlet of the housing.

37. The kit of claim 28 wherein a three-way valve is affixed to the outlet of the housing.

38. The kit of claim 28 wherein a tubing is affixed to the outlet of the housing.
